(19) United States
(12) Patent Application Publication
(10) Pub. No.: US 2007/0105769 A1
(43) Pub. Date: May 10, 2007

(54) Methods of treating tissue defects

(75) Inventor: Bruce Simon, Mountain Lakes, NJ (US)

Correspondence Address: HARNESS, DICKEY & PIERCE, P.L.C., P.O. BOX 828, BLOOMFIELD HILLS, MI, 48303, US

(73) Assignee: EBI, L.P., Parsippany, NJ (US)

(21) Appl. No.: 11/268,699

(22) Filed: Nov. 7, 2005

(51) Int. Cl. A61K 38/18; A61N 1/00

(52) U.S. Cl. 514/12; 607/2

(57) ABSTRACT

Methods of treating tissue defects, including defects such as bone fractures, vertebrae fusions, and spinal disc repair, using electric or electromagnetic fields and growth factors. In various embodiments, the present invention provides methods for the treatment of a human or other mammal subject in need thereof, by administering electric stimulation and growth factors to stimulate endogeneous stem cells in the subject to facilitate healing. Other embodiments include methods of administering electric stimulation, growth factors, and stem cells to the defect. In various embodiments the amount of growth factor is a subefficacious amount.

### INTRODUCTION

The present invention relates to methods for treating trauma, disease, or tissue disorders.

There are a number of complex steps and processes that are involved in the body's response to a tissue defect, such as bone remodeling and healing. One step in the healing of bone defects includes the proliferation of mesenchymal stem cells from the bone marrow, periosteum, and surrounding soft tissue. And unlike most other tissues which heal when injured by forming connective tissue, bone heals by the formation of new bone. Bone is also subject to constant breakdown and re-synthesis in a complex process mediated by osteoblasts, which produce new bone, and osteoclasts, which destroy bone. Osteoblasts arise when mesenchymal stem cells located near bony surfaces differentiate under the influence of growth factors. For example, locally produced bone morphogenic protein (BMP) growth factors can transform stem cells into osteoprogenitor cells. Further differentiation of these cells is an important part of bone repair.

Growth factors are proteins that can affect proliferation and differentiation of various cell types, including stem cells such as mesenchymal stem cells. Certain growth factors have shown clinical benefit in treatment of bone defects, injuries, disorders, or diseases. In particular, the bone morphogenic proteins, including BMP-2 and BMP-7, have shown clinical benefit in the treatment of bone fractures and spine fusions. However, the amount of the bone morphogenic protein required for treatment is large, often requiring several milligrams, such that clinical use is limited by great expense.

Electromagnetic fields can also affect bone repair. Exposure to electric fields can promote connective tissue repair and can accelerate the healing of bone fractures, extracellular matrix synthesis, and the incorporation of bone grafts. For example, the synthesis of cartilage molecules is enhanced by electric stimulation. And furthermore, electric fields can accelerate cell differentiation and stimulate expression of growth factors.

Another response to a bodily trauma, disease, or disorder is the recruitment of pluripotent stem cells to the site of the trauma, disease or disorder. Repair and/or healing are achieved in part by subsequent proliferation and differentiation of the stem cells into specific cells. For example, mesenchymal stem cells proliferate and differentiate into specific cells to repair damage to bone.

Stem cells can be obtained from embryonic or adult tissues of humans or other animals. Irrespective of origin, all stem cells are initially unspecialized cells capable of dividing and renewal, and can give rise by differentiation to specialized cell types (see e.g., National Institutes of Health, Stem Cell Basics, available on the internet at http://stemcells.nih.gov/info/basics/). For example, mesenchymal stem cells, which are stem cells obtained from adult or embryonic connective tissues, can differentiate into many different cell types such as bone, cartilage, fat, ligament, muscle and tendon.

The role of stem cells in certain therapeutic applications has been investigated. See, for example: U.S. Pat. No. 5,197,985, Caplan et al., issued Mar. 30, 1993; U.S. Pat. No. 5,226,914, Caplan et al., issued Mar. 30, 1993; U.S. Pat. No. 5,486,359, Caplan et al., issued Mar. 30, 1993; U.S. Pat. No. 5,811,094, Caplan et al., issued Sep. 22, 1998; U.S. Pat. No. 6,355,239, Bruder et al., issued Mar. 12, 2002; U.S. Pat. No. 6,387,367, Davis-Sproul et al., issued May 14, 2002; U.S. Pat. No. 6,541,024, Kadiyala et al., issued Apr. 1, 2003; and Eppich et al., Nature Biotechnology 18: 882-887, 2000. And therapies involving the alteration of cell or tissue properties by exposure to electromagnetic fields have been proposed. See, Aaron and Ciombor, J. Cellular Biochemistry 52: 42-46, 1993; Aaron et al., J. Bone Miner. Res. 4: 227-233, 1989; Aaron and Ciombor. J. Orthop. Res. 14: 582-589, 1996; Aaron et al., Bioelectromagnetics 20: 453-458, 1999; Aaron et al, J. Orthop. Res. 20: 233-240, 2002; Ciambor et al., J. Orthop. Res. 20: 40-50, 2002; U.S. Pat. No. 6,485,963, Wolf et al., issued Nov. 26, 2002; U.S. Pat. No. 5,292,252, Nickerson et al., issued Mar. 8, 1994; U.S. Pat. No. 6,235,251, Davidson, issued May 22, 2001; and Binderman et al., Biochimica et Biophysica Acta 844: 273-279, 1985.

### SUMMARY

Aspects of the present invention provide for using electric or electromagnetic field stimulation, such as pulsed electromagnetic fields (PEMFs) and growth factors to treat a tissue defect in a human or other mammal subject. In various aspects, the effect of the growth factor treatment is potentiated by the electric field. That is, the effect of the growth factor in addition to the electric field is greater than the effect of either the growth factor or the electric field alone. For example, certain embodiments of the present invention can allow for effective treatment using a growth factor and an electric field where the growth factor is at a subefficacious level.

In various embodiments, the electric stimulation can be a direct current electric field, a capacitatively coupled electric field, or a pulsed electromagnetic fields (PEMF). In various embodiments, the growth factor is a member of the transforming growth factor beta (TGF-β) superfamily, such as a bone morphorgenic protein (BMP).

In various embodiments, the present invention provides methods for the treatment of a human or other mammal subject having a tissue defect, by optionally implanting stem cells into the subject at the site of a defect, and administering electric stimulation and growth factor to the stem cells in situ. In various embodiments, the present invention provides for modulating stem cell activity using electrical stimulation and growth factors where the result is more efficacious than either administering electrical stimulation or growth factors independently of one another. And in various embodiments, the present invention provides for modulating stem cell activity using electrical stimulation and growth factors where the amount of growth factor required to achieve a beneficial result is less than the amount required to obtain a beneficial result without electrical stimulation.

The present invention affords various benefits over the prior art. While not wishing to be bound by theory, such benefits can include one or more of: increased or accelerated proliferation of endogenous stem cells in situ; enhanced differentiation of endogenous stem cells in situ; reduced amounts of growth factors required to achieve beneficial effects; increased rate of healing of tissue defects; and more complete healing of tissue defects.

Further areas of applicability and advantages will become apparent from the following description. It should be understood that the description and specific examples, while exemplifying various embodiments of the invention, are intended for purposes of illustration only and are not intended to limit the scope of the invention.

### DESCRIPTION

The following definitions and non-limiting guidelines must be considered in reviewing the description of this invention set forth herein.

The headings (such as “Introduction” and “Summary,”) and sub-headings (such as “Electric Stimulation”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific examples are provided for illustrative purposes of how to make, use, and practice the methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

The present invention involves the treatment of tissue defects in humans or other animal subjects. Specific materials to be used in the invention must, accordingly, be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

Electric Stimulation:

The term “electric stimulation” as used herein includes exposing stem cells to an electric field, such as a direct current electric field, a capacitatively coupled electric field, an electromagnetic field, a pulsed electromagnetic field (PEMF), or combinations thereof. The term “electric stimulation,” as used herein, does not include an electric or electromagnetic field associated with ambient conditions, such as an electric field generated by casual exposure to radios, telephones, desktop computers or similar devices.

The strength of the electric field produced during electrical stimulation is preferably at least about 0.5 microvolts per centimeter. In various embodiments involving administration of direct current, the current is preferably a direct current of at least about 0.5 microamperes. In various embodiments, the electric stimulation comprises administration of direct current, where the direct current electric field has an intensity of at least about 0.5 microamperes, preferably from about 10 to about 200, and preferably from about 20 to about 100 microamperes. Specific embodiments include those wherein the intensity is about 20, about 60, and about 100 microamperes.

The field may be constant, or varying over time. In various embodiments, the field is a temporally varying capacitatively coupled field. In one embodiment, a sinusoidally-varying electric field is used. For example, various embodiments use a sinusoidally-varying electric field for electrodes placed across tissue at the site of a tissue defect. Such an implantation site can be a human limb, for example. Preferably, such a sinusoidally-varying electric field has a peak voltage across electrodes placed across the cells of from about 1 volt to about 10 volts, more preferably about 5 volts. Preferably, the electric field has peak amplitude of from about 0.1 millivolt per centimeter (mV/cm) to about 100 mV/cm, and more preferably about 20 mV/cm. In various embodiments, the sinusoidal field has a frequency of from about 1,000 Hz to about 200,000 Hz, preferably about 60,000 Hz.

Exposure of tissue defects to PEMFs can be accomplished using methods and apparatus known in the art for exposure of cells and tissues to pulsed fields, for example, as disclosed in the following literature: Binderman et al., Biochimica et Biophysica Acta 844: 273-279, 1985; Aaron et al, Journal of Bone and Mineral Research 4: 227-233, 1989; and Aaron et al., Journal of Orthopaedic Research 20: 233-240, 2002. Parameters of exposure to an electric stimulation field, such as pulse duration, pulse intensity, and numbers of pulses can be determined by a user. In various embodiments, pulse duration of a PEMF can be from about 10 microseconds per pulse to about 2000 microseconds per pulse, and is preferably about 225 microseconds per pulse. In various embodiments, pulses are comprised in electromagnetic “bursts.” A burst can comprise from 1 pulse up to about 200 pulses. Preferably, a burst comprises from about 10 pulses to about 30 pulses, more preferably about 20 pulses. Bursts can be repeated while applying PEMFs. In various embodiments, bursts can be repeated at a frequency of from about 1 Hertz (Hz) to about 100 Hz, preferably at a frequency of about 10 Hz to about 20 Hz, more preferably at a frequency of about 15 Hz. In addition, in some preferred embodiments, bursts can repeat at a frequency of about 1.5 Hz, or about 76 Hz. A burst can have a duration from about 10 microseconds up to about 40,000 microseconds; preferably, a burst can have a duration of about 4.5 milliseconds.

An electric field used herein can be produced using any suitable method and apparatus, including such methods and apparatuses known in the art. Suitable apparatus include a capacitatively coupling device such as a SpinalPak® (EBI, L.P., Parsippany, N.J., U.S.A.) or a DC stimulation device such as an SpF® XL IIb spinal fusion stimulator (EBI, L.P.). A PEMF can be produced using any known method and apparatus, such as using a single coil or a pair of Helmholtz coils. For example, such an apparatus includes the EBI Bone Healing System® Model 1026 (EBI, L.P.). In other embodiments, the electrical stimulation comprises direct current electric field generated using any known device for generating a direct current electric field, such as, for example, an Osteogen™ implantable bone growth stimulator (EBI, L.P., Parsippany, N.J.).

Growth Factors:

The term “growth factor” as used herein includes at least one growth factor or a mixture of more than one growth factor. In various embodiments of the present invention the growth factor can be a member of the transforming growth factor beta (TGF-β) superfamily. Various embodiments of the present invention can include one or more growth factors selected from: VEGF-1, a fibroblast growth factor (FGF) such as FGF-2, epidermal growth factor (EGF), an insulin-like growth factor-1 (IGF) such as IGF-1 or IGF-II, a transforming growth factor (TGF) such as TGF-β, platelet-derived growth factor (PDGF), EGM, and a bone morphogcnetic protein (BMP) such as BMP-2, BMP-4, BMP-6 or BMP-7. Preferred embodiments can include BMP-2 and/or BMP-7.

In various aspects of the present invention, the combination of electric stimulation and growth factor can allow the use of a lower amount of growth factor, where the lower amount of growth factor alone would not obtain an appreciable beneficial result in the absence of the electric stimulation. In various embodiments, methods include administering an electric field and a subefficacious amount of growth factor. A “subefficacious amount” of growth factor is an amount of growth factor that is not efficacious in treating a tissue defect by itself.

In various embodiments comprising the culturing of stem cells with growth factors, a BMP is administered at an amount from about 1 ng/mL to about 100 ng/mL. Additional embodiments include BMP-2 from about 1 ng/mL to about 100 ng/mL. More preferably, various embodiments include BMP-2 from about 10 ng/mL to about 70 ng/mL, and in one embodiment at a level of about 40 ng/mL.

In various embodiments, growth factors are administered to the site of a tissue defect at a level less than the levels that such factors are used clinically without electrical stimulation, preferably of from about 0.5% to about 50% of such clinical levels, more preferably from about 1% to about 30%, from about 2% to about 20%, or from 5% to about 10% of the clinical dose without electrical stimulation. Thus, in some embodiments, a growth factor is administered at a total dose level of from about 0.1 to about 5 mg, preferably from about 0.2 to about 2 mg.

Without limitation to a specific mechanism of action, the present invention provides various embodiments for administration of electric stimulation and growth factors to stem cells in situ, where the electrical stimulation potentiates activity of growth factors. “Potentiates” or “potentiation” of the effect of the growth factors refers to the ability of the electric field to enhance or increase the effect of growth factors at a particular growth factor concentration, where the growth factors at the same concentration would have a reduced or negligible effect on the stem cells in the absence of the electric field.

Stem Cells:

The methods of the present invention can optionally use endogenous and/or exogenous stem cells. Preferably the stem cells are mammalian stem cells and preferably human stem cells. Stem cells can be derived from any known tissue source, for example, cartilage, fat, bone tissue (such as bone marrow), ligament, muscle, synovia, tendon, umbilical cord (such as umbilical cord blood), and embryos. In a preferred embodiment, mesenchymal stem cells are used. In some embodiments, such implanted stem cells are cultured, such as stem cells cultured and in the presence of electrical stimulation as disclosed in U.S. patent application Ser. No. 10/924,241, Simon, filed Aug. 20, 2004, and U.S. patent application Ser. No. ______, Attorney Docket Number 5490E-000344/CPA, Simon, filed concurrently with this application. In other embodiments, implanted stem cells may be obtained by other means, such as by collection from a human or other animal source. In various embodiments, the stem cells may be autologous, that is, collected from the subject to whom they are to be administered. The stem cells may be isolated and concentrated using a variety of methods, including centrifugation, enzymatic, ultrasonic, and other methods known in the art. In various methods, electrical stimulation is applied to endogenous stem cells at the site of the tissue repair.

In various embodiments, the stem cells of the invention are mesenchymal stem cells. For example, the stem cells may be mesenchymal stem cells capable of differentiating into bone, cartilage, vasculature, or blood cells. Also, for example, the mesenchymal stem cells can be osteogenic stem cells, chondrogenic stem cells, angiogenic stem cells, or hematopoietic stem cells. In a preferred embodiment, the mesenchymal stem cells are embryonic or adult mesenchymal stem cells derived from embryonic or adult tissues, respectively, wherein “adult stem cells” include stem cells established from any post-embryonic tissue irrespective of donor age.

In other various embodiments, the stem cells are autologous stem cells. In further various embodiments, the stem cells are allogeneic stem cells. In still other various embodiments, the stem cells are xenogeneic stem cells. Preferably, the stem cells are autologous stem cells or allogeneic stem cells. More preferably, the stem cells are autologous mesenchymal stem cells or allogeneic mesenchymal stem cells. Most preferably, the stem cells are autologous mesenchymal stem cells.

Stem cells utilized in the present invention can be collected, established into cell lines, cultured and propagated in vitro by methods including standard methods among those known in the art. Such methods include those disclosed in U.S. Pat. No. 6,355,239, Bruder et al., issued Mar. 12, 2002; and U.S. Pat. No. 6,541,024, Kadiyala et al, issued Apr. 1, 2003. The stem cells of this invention additionally can also be grown in vitro in a culture medium comprising one or more growth factors, such as VEGF-1, a fibroblast growth factor (FGF) such as FGF-2, epidermal growth factor (EGF), an insulin-like growth factor-1(IGF) such as IGF-1 or IGF-II, a transforming growth factor (TGF) such as TGF-β, platelet-derived growth factor (PDGF), EGM, and a bone morphogenetic protein (BMP) such as BMP-2, BMP-4, BMP-6 or BMP-7.

While not wishing to be bound by theory, the present invention of administering electric stimulation and growth factor can, in various embodiments, modulate endogenous and/or exogenous stem cell activity. As referred to herein, “modulate” refers to the modification of one or more activities of stem cells by, for example, enhancing or increasing such activities. In various embodiments, the activity is one or more of: increased proliferation, enhanced production of molecules normally produced by the stem cells (such as molecular components of the extracellular matrix (ECM)), and accelerated differentiation of stem cells into differentiated cell types. Accelerated differentiation of stem cells includes enhanced production of differentiation markers of differentiated cell types derived from the stem cells, such as specialized ECM markers, wherein “enhanced production” includes increased and/or accelerated production of such markers, as compared to control stem cells that are not subjected to an electric or electromagnetic field but otherwise are under the same conditions.

Methods of Treatment:

The present invention provides methods of treating a human or other mammal subject having a tissue defect, by administering electrical stimulation and growth factors at the site of a tissue defect in a subject. In various preferred embodiments, such methods involve treatment of a tissue defect with electrical stimulation and growth factors, using less growth factor than treatment without electrical stimulation. Accordingly, in preferred embodiments, such methods involve treatment of a tissue defect by using a subefficacious amount of growth factor in conjunction with electrical stimulation.

As referred to herein, such “tissue defects” include any condition involving tissue which is inadequate for physiological or cosmetic purposes. Examples of such defects include those that are congenital, those that result from or are symptomatic of disease or trauma, and those that are consequent to surgical or other medical procedures. Embodiments include treatment for vascular, bone, skin, nerve, and organ tissue defects. Examples of such defects include those resulting from osteoporosis, spinal fixation procedures, hip and other joint replacement procedures, chronic wounds, myocardial infarction, fractures, sclerosis of tissues and muscles, Alzheimer's disease, Parkinson's disease, and spinal cord or other nerve injury.

In various embodiments, the compositions and methods of this invention may be used to repair bone or cartilage defects. A preferred embodiment is for the treatment of bone defects. As referred to herein, such “bone defects” include any condition involving skeletal tissue which is inadequate for physiological or cosmetic purposes. Examples of such defects include those that are congenital (including birth defects), those that result from disease or trauma, and those that are consequent to surgical or other medical procedures. Examples of such defects include those resulting from bone fractures (such as hip fractures and spinal fractures), osteoporosis, spinal fixation procedures, intervertebral disk degeneration (e.g., herniation), and hip and other joint replacement procedures.

In optional embodiments, the present invention provides methods of treating a human or other mammal subject having a tissue defect by implanting stem cells treated with electrical stimulation and growth factors at the site of the tissue defect. In various embodiments, methods comprise administering electric stimulation and growth factors to modulate endogenous stem cells at a tissue defect site. In various embodiments, stem cells are implanted in a culture medium in which they are grown. In other embodiments, stem cells are isolated from a culture medium, and implanted.

In various embodiments, a pharmaceutically-acceptable scaffold is implanted in the human or mammal subject at the site of the tissue defect. As referred to herein, a “scaffold” is a material that contains or supports tissue growth, preferably enabling growth at the site of implantation. In further embodiments, stem cells, growth factors or combinations thereof can be mixed with a scaffold material prior to implantation. In still further embodiments, a scaffold material is implanted either before or after the stem cells are implanted.

Suitable scaffold materials include porous or semi-porous, natural, synthetic or semi-synthetic materials. In various embodiments for the treatment of a bone defect, a scaffold material can be an osteoconductive material. Scaffold materials include those selected from the group consisting of bone (including cortical and cancellous bone), demineralized bone, ceramics, polymers, and combinations thereof. Ceramics include any of a variety of ceramic materials known in the art for use for implanting in bone, including calcium phosphate (including tricalcium phosphate, tetracalcium phosphate, hydroxyapatite, and mixtures thereof. Polymers include collagen, gelatin, polyglycolic acid, polylactic acid, polypropylenefumarate, and copolymers or combinations thereof. Ceramics useful herein include those described in U.S. Pat. No. 6,323,146 to Pugh et al., issued Nov. 27, 2001, and U.S. Pat. No. 6,585,992 to Pugh et al., issued Jul. 1, 2003. A preferred ceramic is commercially available as ProOsteon™ from Interpore Cross International, Inc. (Irvine, Calif., U.S.A.). Growth factors are preferably incorporated into such composition at levels below those known in the art for compositions used without electrical stimulation. In various embodiments, growth factors are present at a concentration of from about 0.01 mg/ml to about 1 mg/ml of scaffold material, preferably from about 0.08 mg/ml to about 0.5 mg/ml, preferably from about 0.1 mg/ml to about 0.3 mg/ml, of scaffold material.

The following non-limiting examples illustrate the methods of the present invention.

### EXAMPLE 1

In a method for enhancing spinal disc repair, the growth factor BMP-2, suspended in a vehicle at 0.1 mg/mL, is injected into a degenerating spinal disc in a human patient. An external device providing a capacitatively coupled electric field, or a PEMF, is then worn by the patient. Exposure of the degenerating spinal disc site to an electric or electromagnetic field produced by the device and conjunctive administration of growth factors stimulates endogenous stem cells to proliferate and differentiate into nucleus cells and annulus disc cells and also increases extracellular matrix production by those cells, leading to disc repair.

In the above example, BMP-4, BMP-6 and BMP-7 are substituted for BMP-2, with substantially similar results.

### EXAMPLE 2

In a method for treating a bone fracture, about 0.5 mg of BMP-7 is injected into the site of the fracture and a pulsed electromagnetic field (PEMF) is applied using a Helmholtz coil. Radiological imaging of the fracture indicates substantial healing of the fracture after four weeks.

In the above example, about 0.2 mg of BMP-2, BMP-4 or BMP-6 is substituted for BMP-7 with substantially similar results.

### EXAMPLE 3

In a method for healing vertebra after posterior lateral spine fusion, bone marrow-derived adult mesenchymal stem cells are mixed with growth factors and osteoconductive granules comprising a calcium phosphate material such as hydroxyapatite, and implanted into a patient. An implantable direct current stimulator is placed internally in the vicinity of the graft to provide an electric field in situ to enhance bone formation. Bone healing is accelerated through this treatment.

In the above example, noninvasive electrical stimulation is effected using an electric or electromagnetic field generating device to apply capacitatively coupled electric fields or PEMFs, with substantially similar results. Also, in the above example, a composition comprising a scaffold material, such as demineralized bone and/or collagen is implanted with the stem cells and growth factors, with substantially similar results.

### EXAMPLE 4

In a method of this invention, a hip fracture is treated with administration of an electric field, growth factor, and implantation of exogenous stem cells. A culture system is used to expand mesenchymal stem cell numbers or generate three-dimensional constructs. In this system, mesenchymal stem cells are derived from muscle, supplied with medium containing growth factors, and grown in culture dishes placed between pairs of Helmholtz coils to generate a uniform PEMF. The stem cells are then harvested, and mixed with collagen as a scaffold material. The composition is then implanted at the site of the fracture, and electric stimulation is continued in situ, thereby accelerating healing of the bone.

In the above example, the stem cells, supplied with medium containing growth factors, are grown in culture dishes placed within a capacitatively coupled electric field, with substantially similar results. Also in the above example, the stem cells are derived from bone marrow, muscle, fat, umbilical cord blood, or placenta, with substantially similar results. Also in the above example, collagen is replaced with polyglycolic acid or polylactic acid, with substantially similar results.

### EXAMPLE 5

In a method of this invention, the differentiation of mesenchymal stem cells is enhanced by administration of PEMF and growth factor BMP-2. Human mesenchymal stem cells are plated in culture dishes such as, for example, 10 cm² culture dishes, and the non-differentiating cultures are grown to near confluence. The cells in the dishes are then stimulated to undergo osteoblast differentiation in the presence or absence of PEMFs and growth factor BMP-2, where BMP-2 is added to the medium to make 40 ng/mL. Samples are taken at different times throughout the differentiation process and examined. Day of plating is designated as day −2. At day 0 (2 days later), cells are stimulated down the osteoblast differentiation pathway. Osteoblast differentiation (to mineralization in vitro) is induced with osteoblast medium (Mesenchymal Stem Cell Growth Medium/10% Fetal Bovine Serum/0.1 M dexamethasone/50 μM ascorbate/10 mM β-glycerophosphate/50 ng/mL BMP-4). Cell numbers and extracts are collected at days 0, 1, 2, 6, 9, 12, 14, 21, and 28 following mineralization stimulus. Some cells are stained for mineralized state, others are used for Western blot, RNA extraction, osteocalcin assays, and alkaline phosphatase assays. The stem cells subjected to both PEMF and BMP-2 show increased proliferation compared to stem cells subjected to only BMP-2, as evidenced by increased incorporation of ³²P dCTP, as well as increased differentiation of osteoblasts, as evidenced by increased amounts of osteocalcin and alkaline phosphatase, as well as various osteocalcin mRNAs detected using Northern blot analysis. Likewise, stem cells treated with PEMF and BMP-2 show increased proliferation compared to control stem cells not subjected to either PEMF or BMP-2.

In this example, the culture matrix, comprising stem cells and BMP-2, is mixed with osteoconductive granules, and implanted at the site of a spinal fusion surgery. PEMF is then applied to the fusion site. Radiographic imaging indicates substantial fusion of the vertebra within six weeks.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

What is claimed is:

1. A method of treating a tissue defect in a human or other mammal subject comprising administering to the site of said defect an electric stimulation and a growth factor.

2. The method according to claim 1, wherein said electric stimulation comprises an electric field selected from the group consisting of a direct current electric field, a capacitatively coupled electric field, and a pulsed electromagnetic field.

3. The method according to claim 2, wherein said electric stimulation is a pulsed electromagnetic field.

4. The method according to claim 1, wherein the growth factor is a member of the TGF-β superfamily.

5. The method according to claim 4, wherein the growth factor is a bone morphogenic protein (BMP).

6. The method according to claim 4, wherein said BMP is administered at a level of from about 0.2 to about 2 mg.

7. The method according to claim 1, wherein the growth factor is selected from the group consisting of VEGF-1, FGF-2, EGF, IGF-1, TGF-β, PDGF, IGF-I, IGF-II, EGM, BMP-2, BMP-4, BMP-6, BMP-7, and mixtures thereof.

8. The method according to claim 1, wherein said defect is a bone defect.

9. The method according to claim 8, further comprising implanting a scaffold material at said site.

10. The method according to claim 9, wherein the scaffold material is selected from the group consisting of bone, demineralized bone, ceramics, polymers, and combinations thereof.

11. The method according to claim 10, wherein the scaffold material comprises osteoconductive granules comprising hydroxyapatite.

12. The method according to claim 1, wherein said defect is a bone disease, fracture, wound, injury, birth defect, spinal fusion, defective cartilage, a site of an orthopedic implant, a degenerated or herniated intervertebral disk, a site of intervertebral disk replacement, or a spinal cord injury.

13. The method according to claim 1, wherein said growth factor is administered at a subefficacious level.

14. The method according to claim 13, further comprising administering stem cells to the site of said defect.

15. The method according to claim 15, wherein said stem cells are mesenchymal stem cells.

16. A method of treating a bone fracture in a human or animal subject comprising administering to the site of said defect a pulsed electromagnetic field and a BMP at a level of from about 0.2 to about 2 mg.

17. A method of treating a bone fracture in a human or animal subject according to claim 17, further comprising administering to the site of said defect mesenchymal stem cells.

18. A method according to claim 17, further comprising administering to said site an osteoconductive scaffold material.
